(19) United States
(12) Patent Application Publication
(10) Pub. No.: US 2026/0049351 A1
(43) Pub. Date: Feb. 19, 2026

(54) COMPOSITIONS AND METHODS FOR AMPLIFYING LONG NUCLEIC ACID MOLECULES

(71) Applicant: Fluid Discovery, San Francisco, CA (US)

(72) Inventor: Adam Abate, San Francisco, CA (US)

(21) Appl. No.: 19/242,521

(22) Filed: Jun. 18, 2025

Related U.S. Application Data
(60) Provisional application No. 63/661,184, filed on Jun. 18, 2024.

(51) Int. Cl. C12Q 1/6844

(52) U.S. Cl. CPC C12Q 1/6844

(57) ABSTRACT

Provided herein are compositions and methods for amplifying long nucleic acid molecules. In particular, provided herein are compositions and methods for amplifying long nucleic acid molecules that are mixed in complex samples, for example, complex biological samples.

### CROSS-REFERENCE TO RELATED APPLICATIONS

The present application claims priority to U.S. Provisional Patent Application No. 63/661,184, filed Jun. 18, 2024, which is incorporated by reference in its entirety.

### FIELD

Provided herein are compositions and methods for amplifying long nucleic acid molecules. In particular, provided herein are compositions and methods for amplifying long nucleic acid molecules that are mixed in complex samples, for example, complex biological samples.

### BACKGROUND

Detection and analysis of long nucleic acid molecules poses challenges. When such molecules are present in a sample at low concentrations or copy number, amplification may be needed to facilitate detection and analysis. Amplification of long nucleic acid molecules, particularly in complex samples, presents challenges compared to the amplification of shorter molecules. For example, long nucleic acid targets may exist in multiple copies, have sequence homology with other targets in the same sample, and may be present amidst a significantly larger quantity of non-target background nucleic acids. Traditional amplification methods such as the polymerase chain reaction (PCR) often fail in such scenarios for multiple reasons: different targets may amplify at varying rates, rare targets can be challenging to amplify reliably, and targets of different lengths and sequence characteristics may amplify differently. This often results in the amplification of only the fastest-amplifying, and often least interesting, targets. New approaches are needed.

### SUMMARY

Provided herein are compositions and methods for amplifying long nucleic acid molecules. In particular, provided herein are compositions and methods for amplifying long nucleic acid molecules that are mixed in complex samples, for example, complex biological samples.

In some embodiments, the technology comprises compositions, systems, or methods for amplifying long target DNA in a sample comprising background DNA.

In some such embodiments, methods comprise: a) partitioning DNA from a sample into a plurality of partitions such that, on average, there is one copy of a long target DNA in a partition containing the long target DNA and wherein the partition further comprises background DNA; and b) amplifying DNA in the plurality of partitions using one or more long target DNA- specific primers under conditions that saturate the partitions containing long target DNA such that all long target DNA in different partitions reach similar endpoint concentrations irrespective of amplification rate. The amplicon products of such a reaction are analyzed (e.g., sequenced).

Any type of nucleic acid molecule may be used (e.g., DNA, RNA, cDNA, etc.) from any source (e.g., cells, virus, bacteria, environmental samples, biological samples, etc.). In some embodiments, the long target nucleic acid is greater than or equal to 1000 bases or base pairs long (e.g., equal to or greater than 2000, 3000, 4000, 5000, 6000, 7000, 8000, 9000, 10000, 12000, 15000, 20000, 30000, 40000, 50000, . . . ; or any value therein between). In some embodiments, the nucleic acid is purified prior to partitioning and/or amplification. In some embodiments, the nucleic acid is modified (e.g., via addition or generation of a Modified Base Identifiers (MBI)) prior to partitioning and/or amplification.

In some embodiments, methods comprise analyzing target nucleic acid molecule and sequences flanking the target nucleic acid molecules using one or more or each of the steps of: a) fragmenting nucleic acids from a sample such that at least a sub-population of fragments contain target sequence (e.g., full target sequence) with a portion of flanking sequence still attached to the target sequence (e.g., flanking sequence attached to each end of full target sequence), b) attaching dumbbell primers to the fragments, thereby generating circularized fragments comprising a single stranded loop (e.g., with partial homology between the primers), c) encapsulating the circularized fragments into partitions, d) amplifying the loops using primers complimentary to portions of the target sequence, thereby generating amplification products that comprise portions of the loops comprising target sequence, flanking regions, and/or loop primers, and e) analyzing amplicons products so as to obtain information about the target sequences and their flanking regions.

Further provided herein are kits comprising components (e.g., reagents) sufficient, necessary, or useful for practicing any of the methods described herein.

Further provided herein are reaction mixtures comprising one or more a target nucleic acids, or modified versions thereof or products generated therefrom, and/or one or more background nucleic acids, or modified versions thereof or products generated therefrom, combined with reagents, as provided in any of the methods described herein.

### DEFINITIONS

As used herein, a “nucleic acid” or “nucleic acid molecule” generally refers to any ribonucleic acid or deoxyribonucleic acid, which may be unmodified or modified DNA or RNA. “Nucleic acids” include, without limitation, single-and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene.

In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a sequence refers to a sequence that has the characteristics of a sequence isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

“Amplification” is a special case of nucleic acid replication involving template specificity. It is to be contrasted with non-specific template replication (e.g., replication that is template-dependent but not dependent on a specific template). Template specificity is here distinguished from fidelity of replication (e.g., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties). Amplification can also be via isothermal approaches, including, but not limited to: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

The term “amplifiable nucleic acid” refers to a nucleic acid that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, may be combined with other nucleic acids or molecules. For example, an isolated nucleic acid may be present in a host cell in which into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, the terms “patient” or “subject” refer to organisms to be subject to various tests provided by the technology. The term “subject” includes animals, preferably mammals, including humans.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc.

### DESCRIPTION OF FIGURES

FIG. 1 depicts improved droplet stability when running ddLA-PCR with SuperFi II by adding stabilizing additives to the buffer after an amplification reaction of full length HIV genome. All cases used SuperFi II reaction mix, the manufacture's recommended thermocycling, and 35 cycles. As shown in the left column of FIG. 1, when using SuperFi II buffer alone in ddLA-PCR reactions, droplets were prone to coalescence or complete catastrophic de-emulsification during the thermocycling process. Droplet coalescence or de-emulsification were remedied by adding a combination of stabilizing additives of PEG 6k MW and TWEEN20 to the SuperFi II as shown in the right column of FIG. 1. NTC=no template control in bulk. Bulk=standard LA-PCR reaction. Droplet=ddLA-PCR conditions.

FIG. 2 shows optimization of the PCR reaction mix with modifications of the manufacturer's recommended “traditional/generic” PCR mix by altering the primer and thermocycling parameters, thereby demonstrating that known inherent thermal degradation during amplification was offset by decreasing the denaturation temperature. All ladder marks are in kbp. All images from 1.2% agarose, it was noted that longer running times cause a gel-shift of the apparent mass. Panel A) Primer titration is in bulk. Panel B) Primary amplification primers demonstrate robust PCR across a range of anneal temperatures (Tₘ). Panel C) The ddLA-PCR reaction could support up to 50 cycles PCR cycles of amplification, though yield gains are not exponential.

FIG. 3 shows a multiparameter analysis of PCR reaction mix with modification of the manufacturer's recommended “traditional/generic” PCR mix by altering the primer and the dNTP parameters. Reactions are in bulk (B), droplet (d), or shaken emulsion (s) conditions. Numbers represent fold changes relative to SuperFi II manual. A reduction of smear products in original droplet condition and band intensity increase are noted. All ladder marks are in kbp.

FIGS. 4A-4B demonstrate the results of the optimized alterations of the PCR mix when applied to a series of mixture experiments with truncated HIV. Amplification of truncated HIV demonstrated low drop out results in spite of high molecular weight and low concentrations. All ladder marks are in kbp. A) In the first experiment, a PCR of 6 kbp, 3 kbp, and 1 kbp provirus plasmids in a ratio of 1:1:1 was performed in bulk and in droplet. The bulk cases show a heavy preference for the smaller amplicons and larger amplicons either are dropped out or are indistinguishable from background noise. The droplet cases show a clear, defined signal for the largest amplicons B) Clear bands are identified for the larger amplicons with another experiment that used a ratio of 1:1:1 of 10 kbp, 6 kbp, and 3 kbp provirus plasmids.

FIGS. 5A-5C show that the modified ddLA-PCR protocol can achieve full length amplification of HIV using real-world starting concentrations of an HIV template based on super loading of the droplets in a manner similar to what is done in well plate contexts. The figures demonstrate the results of a series of loading experiments in which the number of HIV copies within the reaction, or the amount of background DNA was varied. All experimental replicates show amplification. A) The dilution series of HIV ranging from 10,000 replicates per reaction (10,000 JLAT cells per reaction) down to 10 replicates per reaction (10 JLAT cells per reaction). B) HIV provirus amplification reaction is shown in the presence of additional background genomic DNA from uninfected cells. Positive amplification of HIV provirus is identified in the presence of 20,000, 100,000, and 200,000 cells-worth of material, corresponding to an excess of 120 ng of background DNA. C) The graphs confirm the qualitative results from measuring human loci and HIV genome abundance with a multiplexed qPCR analysis, along with showing titration and effect of different emulsification methods. The graphs demonstrate that pipetting produced the most homogeneous distribution and most effective preservation of the integrity of DNA length within droplets.

FIG. 6 shows an evaluation of high-molecular-weight (HMW) DNA degradation in relevant buffer systems, specifically demonstrating SuperFi II Buffer as an effective storage system without further augmentation by showing no appreciable degradation after at least a week. 0=deionized water for gDNA/SuperFi II buffer for 1° PCR after de-emulsification with a static gun. S=supplementation of base with a concentrate that leads to a final additional of 10 mM TRIS pH 9.0 & 1 mM EDTA. P=washing with perfluorooctanol for de-emulsification. Genomic DNA (gDNA) represents material isolated directly from cells using a New England BioLabs HWM DNA isolation kit and sheered to a similar size as amplicon. 1° PCR represents amplicons generated from template JLAT gDNA. All samples were stored at 4° C., with aliquots taken at indicated time.

FIGS. 7A-7B show isolation and amplification of full-length HIV from patient samples using ddLA-PCR. A) Two patient samples, labeled “A” and “B.” The demonstration on the left in FIG. 7A shows isolation of the high molecular weight DNA that was performed on the two patient samples. The middle demonstration of FIG. 7A shows that thermocycling degrades the genomic DNA in the absence of productive PCR. Finally, the demonstration on the right in FIG. 7A shows that sample “A” isolation identified a strong band indicative of target full length HIV amplification, and sample “B” generated a weaker, but broader distribution of amplification. B) qPCR with probes against HIV was used to confirm HIV amplification in sample “B” which was measured at in amplification by approximately 9 Ct (corresponding to approximately 512,000-fold increase).

### DETAILED DESCRIPTION

Provided herein are compositions and methods for reliably amplifying long nucleic acid (e.g., DNA) targets that are mixed in a complex sample. These targets may exist in multiple copies, have sequence homology with other targets in the same sample, and may be present amidst a significantly larger quantity of non-target background DNA.

In some embodiments, the long nucleic acid targets are equal to or greater than 1000 bases or base pairs in length (e.g., equal to or greater than 2000, 3000, 4000, 5000, 6000, 7000, 8000, 9000, 10000, 12000, 15000, 20000, 30000, 40000, 50000, . . . ; or any value therein between). In some embodiments, long nucleic acid targets are equal to or shorter than 200000 bases or base pairs (e.g., equal to or shorter than 100000, 90000, 80000, 70000, 60000, 50000, 40000, 30000, 20000, 10000, . . . ; or any value therein between). Thus, in some embodiments, the long nucleic acid targets may be in a size range bounded by any such values (e.g., 1000-200000, 1000-100000, 1000-50000, 2000-200000, 2000-100000, 10000-50000, etc.).

The long nucleic acid targets may be derived from any source. In some embodiments, long nucleic acid targets are of viral origin and may be found in biological (e.g., a fluid sample from human subject) or environmental samples. In some embodiments, long nucleic acid targets are obtained from cells. In some embodiments, long nucleic acid targets are synthetic molecules. In some embodiments, long nucleic acids are initially found in a compartment or carrier (e.g., cell, capsid, exosome, etc.) and are removed from the compartment or carrier before undergoing further processing or analysis. In some embodiments, the target nucleic acid, and potentially non-target nucleic acid, is purified before undergoing further processing or analysis. To maintain the integrity of long nucleic acid molecules, process steps are selected to avoid unnecessary fragmentation (e.g., selection of appropriate temperatures, use of appropriate pipetting techniques, work in nuclease-free environments, etc.).

The technology provided herein addresses the challenges and limitations of prior approaches by, for example, employing a compartmentalization approach, allowing for the quantitative amplification of all target nucleic acid (e.g., DNA). Specifically, in some embodiments, the sample is compartmentalized into chambers where targets are present at a rate of one copy per compartment. Although background DNA may also be abundant in each compartment, it does not interfere with the targeted amplification because it is not specifically targeted by the primers. Thus, in some embodiments, the approach is not a true digital approach because there may be significantly more than one nucleic acid molecule per partition.

The technology functions in any of a wide variety of partition-based approaches. Examples include droplet-based partitioning (e.g., ddPCR) where, for example, sample is mixed with oil to form an emulsion, creating large numbers of drops, each acting as an individual reaction chamber. Advantages include high partition numbers, robust and reproducible results, and compatibility with high-throughput analysis. Microfluidic chip-based partitioning may also be used. For example, microfluidic chips or arrays with a large number of tiny wells or channels can provide the appropriate partitions. Advantages include precise control over partition size, large-scale partitioning, and minimal sample loss. Nanowell arrays may also be used, where nucleic acid sample is loaded onto a surface containing an array of nanowells. Advantages include high partitioning accuracy, stable reaction environment, and suitability for low-volume samples. Bead-based partitioning may also be used, where nucleic acid molecules are attached to beads and then emulsified in oil to form droplets. Advantages include compatibility with high-throughput sequencing and analysis and the ability to easily recover beads for other downstream processing. Partitions can be generated using any suitable approach, including, but not limited to vortexing, particle templated emulsification, etc. Partitions may be substantially different (i.e., polydispersed) or similar (i.e., monodispersed) in volume.

The technology finds use with a wide range of partition/compartment sizes and volumes. In some embodiments, the compartments (e.g., droplets, wells, etc.) have volume from 1 picoliter to 1 milliliter (e.g., 1 pL, 1 nL, 1 μL, 1 mL; and any range therein between). In some embodiments, there are from 10 to 10¹² compartments or more (e.g., 10, 100, 1000, 10⁴, 10⁵, 10⁶, 10⁷, 10⁸, 10⁹, 10¹⁰, 10¹¹, 10¹², etc.).

The reaction is then subjected to amplification. For long targets, in some embodiments, PCR methodologies are utilized to ensure complete amplification of target molecules within their respective compartments. This results in a product that can be readily analyzed downstream. In some embodiments, the resulting amplicons are sequenced using long-read sequencing techniques or other suitable methods, thereby providing the sequences of the various targets of interest. Suitable long-range sequencing approaches include, but are not limited to, single- molecule real-time (SMRT) sequencing (Pacific Biosciences), Oxford Nanopore Technologies (ONT), linked-read sequencing, synthetic long-read sequencing, and optical mapping.

Importantly, this technology provides a way to overcome the challenges of amplification bias, particularly when aiming to amplify rare, long molecules in a complex background. By maintaining a constant amplification factor for all targets, irrespective of length or other rate-biasing features, this technology allows for comprehensive detection of the full range of target diversity in the original sample. HIV is used in the example section below to illustrate the value of such approaches. For applications like HIV genome sequencing, where HIV genomes may constitute a minuscule fraction of the sample, this approach ensures that the resulting product is not dominated by fast-amplifying, deletion mutants that are often irrelevant to the disease.

In some embodiments, the technology additionally provides compositions and methods for obtaining information that extends beyond the target DNA regions. For example, in some embodiments “Looping PCR” is employed. With looping PCR, the sample DNA is circularized by the addition of hairpin primers. These primers produce a loop of single-stranded DNA that forms a duplex along the original DNA fragment. Through the careful selection of primers, amplicons are generated that not only include the target sequences but also regions of DNA extending beyond the target.

In some embodiments, this is accomplished using “Inversion PCR”-like approaches, allowing for the specific enrichment of target DNA regions from a large background of non-target DNA. The generated amplicons are then subjected to additional long-range sequencing methods to obtain sequence reads that contain both the target and its flanking regions.

This approach is particularly valuable because it allows for the isolation of sequences adjacent to the target region, which are often unknown and therefore untargetable by conventional methods. Furthermore, this approach is insensitive to amplification rate differences that may occur due to sequence features, such as target length. The technology offers a versatile approach for comprehensive genomic studies, capturing not only the targets but also their genomic context, which can be important for a deeper understanding of their function and regulation.

In some embodiments, the technology also employs Modified Base Identifiers (MBIs) in tandem with Long-Range Compartment PCR (LRC-PCR) to augment its capabilities. MBIs serve to label both the target sequence and its adjacent DNA regions with a unique spectral signature that is independent of the target sequence itself. This spectral signature is distributed across the regions containing the target at a density that can be controlled. Following MBI labeling, the target sequences undergo LRC-PCR, generating amplicons that carry this unique MBI spectrum. These amplicons are then sequenced, either in their entirety or through short-read sequencing methods. The MBI labeling serves several important functions: they help in normalizing for PCR duplicates, corrects errors, and facilitate the assembly of long consensus sequences based on overlaps between reads. This use of MBIs adds a layer of sophistication to LRC-PCR, allowing for more precise sequence analysis and data interpretation. The combination of these techniques enhances the ability to differentiate sequences with high similarity and contributes to the generation of highly accurate, long-read sequences, even when starting from complex genomic samples. Examples of MBIs include, but are not limited to, 5-Methylcytosine (5mC), 5-Hydroxymethylcytosine (5hmC), N6-Methyladenine (6mA), N6,2′-O-Dimethyladenosine (m6Am), N1-Methyladenine (1mA), pseudouridine (Y), 5-Formylcytosine (5fC), 5-Carboxylcytosine (5caC), N7-Methylguanosine (m7G), N2-Methylguanosine (m2G), and 2′-O-Methylated Nucleotides.

In some embodiments, the technology employs linear molecule amplification. For example, in some embodiments, the technology provides compositions and methods for reliably and quantitatively amplifying multiple target nucleic acids in a single sample with different amplification rates mixed in a complex background. In some embodiments, the approach comprises one or more or each of the steps of: a) preparing nucleic acids for encapsulation or partition, while maintaining the nucleic acids above a length suitable to fully contain the target sequence(s) of interest, b) mixing the prepared nucleic acids with reagents suitable for amplification, c) encapsulating/partitioning the nucleic acids in compartments at a concentration such that the desired target molecules are at limiting dilution even though the background molecules may not be, thereby ensuring that a majority of the target molecules are able to amplify in independent compartments without competition between them, d) subjecting the sample to conditions so as to stimulate specific amplification of the targets, wherein conditions such as temperature, buffer components, enzyme type, etc., are specified so as to minimally fragment the target nucleic acid through the amplification and yield efficient long ranged amplification; and amplifying sufficiently so as to saturate the compartments containing targets such that all targets reach similar endpoint concentrations irrespective of amplification rate due to, for instance, differences in sequence, structure, length, etc., thereby producing a sample of amplicons of the targets wherein the proportions of the different amplicons are similar to the initial proportions of the original target molecules, thereby generating a quantitative amplification even when targets may have different intrinsic amplification rates, and e) recovering and analyzing the nucleic acids to obtain information about the original targets, such as target concentration and/or sequence. In some embodiments, saturation is achieved where amplification is run to endpoint, such that the amplicons within the target droplets saturate to a final concentration that no longer appreciably increases with additional cycles, thereby yielding amplicon products of the different targets in proportions that are substantially related to the original target concentrations, and thus yielding a quantitative amplification.

The volume and/or number of the partitions employed can be selected so to control the amplicon yield. For example, the partition volume is increased to generate more amplicons of each target at endpoint saturation of the reaction.

Reaction conditions for amplification are selected to avoid fragmentation of long target nucleic acid, while allowing complete amplification of long sequences and amplification saturation (e.g., maximizing yield without exhausting reagent). Such parameters include selection of cycle times, cycle numbers, temperature selection, reagent selection, and reagent concentrations (e.g., primer/dNTP concentration (to get maximum yield)). In some embodiments, denaturation temperatures are from 95 to 90° C. Polymerase enzymes are selected that efficiently polymerize amplicons with high fidelity (e.g., Phusion DNA polymerase, Q5 high-fidelity DNA polymerase, Pfu DNA polymerase, KOD DNA polymerase, Herculase II Fusion DNA polymerase, Platinum SuperFi DNA polymerase, KAPA HiFi DNA polymerase, etc.). Reagent compositions are selected so as to provide efficient and accurate amplification of the targets, such as including additives that increase reaction efficiency (e.g., Super Fi buffer (PEG/Tween+FC40, for droplet stability); Super Fi buffer (propanol diol, for lower denaturation temp).

Amplicon can be analyzed by any suitable technique to obtain the information desired by the user. Such techniques include, but are not limited to, capillary electrophoresis, bioanalyzer, gels, etc., to obtain concentration and/or length information, and in which specific lengths may be selected for analysis, for example, to bias the analysis to specific lengths. In some embodiments, the analysis is nucleic acid sequencing.

In some embodiments, the technology employs looping molecular amplification to allow for analysis of a target sequence as well as sequencing flanking the target sequence. In some such embodiments, the method comprises one or more or all of the steps of: a) fragmenting the nucleic acids such that the fragments often contain whole targets with a portion of the flanking sequence still attached, b) attaching “dumbbell” primers to the fragments, thereby generating a single stranded loop with partial homology between the primers forming a “dumbell” like product, c) encapsulating the circularized fragments into partitions, d) amplifying the loops using primers complimentary to portions of the target sequence, thereby generating amplification products that comprise portions of the loops comprising target sequence, flanking regions, and/or looping primers, and e) recovering and analyzing the products so as to obtain information about the target sequences and their flanking regions in a way that allows specific targets to be associated with specific flanking regions.

In some embodiments, fragmentation is conducted in the presence of fragmenting enzymes. Such enzyme include, but are not limited to fragmentase, Tagmentase (Tn5 transposase), NEBNext® dsDNA Fragmentase, restriction enzymes, and the like. In some embodiments, fragmentation is conducted chemically or mechanically (e.g., heating, shearing, sonication, etc.).

In some embodiments, fragmentation and circularization can occur in a single-step reaction using tagmentation to fragment nucleic acid in a sample, while also adding hairpin primers.

In some embodiments, unique molecule identifiers (UMI) are introduced as part of the hairpins so as to introduce a unique combination of UMIs to each fragment that can be used to identify the fragment and its amplification products to enable for example duplex sequencing and thus allow reduced error rates. In some embodiments, barcodes are introduced, that may provide unique or non-unique identification sequences. In some embodiments, a combination of endogenous sequence information (e.g., fragment ends) and exogenous sequence information (e.g., ligated barcodes) is used to identify molecules. In some embodiments, UMIs or barcodes introduced into the respective ends of each fragment are the same sequence. In some embodiments, flanking sequences can be used, alone or in combination with UMIs, barcodes, or other endogenous sequences, to identify molecules and for error avoidance or correction. In some embodiments, the flanking sequences are used to associated single stranded sequence reads as having been derived from the same original target nucleic acid (e.g., duplex sequence) so as to provide a basis for error correction and to increase the accuracy of sequencing.

A number of different approaches may be used to amplify all or a portion of the target and/or flanking sequences. In some embodiments, inversion PCR (aka inside-out PCR) (see e.g., Ochman, H.; Gerber, A. S.; Hartl, D. L. (1988). “Genetic applications of an inverse polymerase chain reaction.” Genetics. 120(3): 621-623), herein incorporated by reference in its entirety) is employed to amplify the flanking sequences that have unknown sequences and are not amenable to target-specific priming. In some embodiments, a looped target is amplified using primers targeting different regions of the target, such as the target, flanking regions, and introduced primers.

MBIs may be employed in any of the methods. In some embodiments, MBIs are used to distinguish and associate reads of otherwise indistinguishable sequence, to normalize amplification duplications, and to facilitate sequence assembly to generate long consensus sequences of long and or looped targets and their amplicons. For example, MBIs are employed to label DNA sequences with a distinct array of modified bases, which are decoded during the sequencing process. The density of these modified bases is adjustable, creating a unique signal pattern across the sequence. This pattern helps distinguish one sequence from others that may have identical nucleotide composition otherwise. Such unique MBI patterns facilitate the grouping of sequencing reads according to their molecule of origin. This, in turn, allows for the reduction of sequencing errors through consensus generation. Alternatively, overlaps in MBIs can be utilized to concatenate reads from longer sequences, thereby building a full-length consensus sequence using methodologies similar to standard sequencing assembly. The integration of MBIs is particularly advantageous in the context of the compartmentalized PCR techniques described herein. It allows the aggregation of short, overlapping sequences into longer, more reliable sequences.

Also provide herein are compositions and systems (e.g., reaction mixture, kits, reagent compositions, instruments, software) that finds use in conducting any of the methods described herein. For example, in some embodiments, compositions, kits, and systems may comprise reagents (e.g., target-specific primers, dumbbell primers, polymerases, amplification reagents, ligases, sequencing reagents, buffers, controls, MBIs, etc.) for conducting a method described herein. In some embodiments, the kits contain one or more reagent necessary, sufficient, or useful for conducting a method described herein. Also provided are reactions mixtures containing the reagents and one or more nucleic acid molecules from a sample and/or product generated therefrom (e.g., amplicons). Further provided are master mix reagent sets containing a plurality of reagents that may be added to each other and/or to a test sample to complete a reaction mixture.

The compositions and methods described herein find use in any of a wide variety of applications where the analysis of long sequences is important. Such applications include, but are not limited to, structural variation detection (e.g., identifying large insertions, deletions, inversions, translocations, and complex structural variations), de novo genome assembly, synthetic biology, resolving repetitive regions (e.g., sequencing regions with high repeat content such as centromeres, telomers, and transposable elements), haplotype phasing, metagenomic and microbiome studies, human genomics and personalized medicine, epigenetics and methylation analysis, cancer genomics, transcriptomics and isoform sequencing, comparative genomics, pathogen analysis, and the like.

An exemplary embodiment is illustrated in the examples below related to full length HIV genome sequencing. The technique is designed to characterize the genomic diversity of the proviral HIV population in individuals undergoing antiretroviral therapy (ART). The method begins by isolating DNA from the patient's cells where proviral HIV is present. This DNA is then extracted and fragmented to lengths that are sufficiently large to encompass a significant proportion of intact viral genomes. Subsequent to fragmentation, the DNA sample is encapsulated into droplets, and long-range PCR amplification is performed using HIV-specific primers. This ensures that only the targeted HIV genomes are amplified for analysis. Finally, the amplified genomic fragments, or amplicons, are recovered and subjected to sequencing. This sequence data is then analyzed to provide a comprehensive view of the proviral HIV population within the patient, thereby offering valuable insights for therapeutic intervention and management.

In a related embodiment, the method is extended to yield not just the full-length viral genome but also the site of integration within the human genome. This is achieved by circularizing the long DNA fragments containing the viral genome using specialized “dumbbell” primers that form hairpins at the ends of the DNA. This results in a single-stranded loop, wherein the DNA duplex consisting of the HIV genome is physically linked in a loop structure. Notably, there is significant homology between the hairpins, which can be introduced via methods like tagmentation or ligation. Subsequent PCR amplification targets segments of this looped structure. Specifically, primers can be designed to target portions of one strand of the HIV genome, looping across the adjacent human genomic DNA and the hairpin primers. This produces amplicons that not only contain the HIV genome but also the flanking human genomic DNA at the integration site. These amplicons are then sequenced, allowing for the direct mapping of the HIV genome sequence to its integration junctions within the human genome. This provides a more comprehensive analysis, enhancing our understanding of both the viral genome and its interaction with the host genome.

In another embodiment, Long-Range Compartment PCR (LRC-PCR) is combined with Modified Base Identifiers (MBIs) for an efficient and cost-effective sequencing approach to investigate the HIV reservoir along with paired human junction information. In this setup, human genomic DNA is labeled with modified bases, for instance, through the addition of a methylase enzyme that randomly methylates the DNA. A subsequent step introduces methyl deaminase, facilitating a cytosine-to-thymine (C-T) transformation during the PCR amplification process. An initial PCR is performed using HIV-specific primers, aiming to amplify the viral genomes. A second PCR focuses on amplifying the junctions between the HIV and human DNA. For this purpose, one primer targets the HIV genome while the second primer targets the tagmented region of the human DNA. These primers are designed to produce amplicons that include enough of the HIV genome to specify a unique MBI for that particular genome. This MBI will also be present in the corresponding HIV genome amplified in the previous PCR step. The material from both amplifications is then sequenced. Post-sequencing, the junctions is paired to the corresponding HIV genomes based on overlapping MBIs. This integrated approach allows for robust, precise, and cost-effective characterization of the HIV reservoir, delivering both the viral and host genomic information in a single workflow.

### EXAMPLES

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure and should not be viewed as limiting to the scope of the disclosure. The present disclosure has multiple aspects, illustrated by the following non-limiting examples.

### Example 1: DNA Preparation for Full Length HIV Digital Droplet PCR

### 1. Extraction of Ultra-High Molecular Weight DNA Using Fresh or Frozen Cells

Reagents were organized according to steps in Monarch UHMW DNA Extraction Kit for Cells & Blood (t3050) where 2 mL tubes (DNA low-bind, round bottom tubes) at room temperature were acquired; Nuclei Prep Buffer and RNAse A were placed on ice; Nuclei Lysis Buffer and Proteinase K were placed on ice; room temperature precipitation enhancer, DNA capture beads, and isopropanol were acquired; and room temperature wash buffer, Elusion Buffer I, and Bead Retainer were acquired.

Fresh cells were placed on ice. Frozen cells were thawed and placed on ice. Cell concentrations were determined by using a hemocytometer. A sample of 0.5-2 million cells were aliquoted into 2 mL tubes (DNA low-bind, round bottom, room temperature). The aliquoted cells in the 2 mL tubes were each centrifuged at 300 rcf for 2 minutes. The supernatant was removed from each without disturbing the pellet.

The Nuclei Prep Solution was prepared by combining 150 μL Nuclei Prep Buffer and 5 μL RNAse A for each cell aliquot (2 mL tube). 150 μL Nuclei Prep Solution was then added to each aliquot (2 mL tube) and gently pipetted 10 times to resuspend cells in the Nuclei Prep Solution followed by incubation at room temperature for 2 minutes.

The Nuclei Lysis Solution was then prepared by combining 150 μL Nuclei Lysis Buffer and 10 μL Proteinase K for each cell aliquot (2 mL tube). 150 μL Nuclei Lysis Solution was then added to each aliquot (2 mL tube) which was subsequently inverted 10 times to mix, followed by an incubation period of 10 minutes at 56° C.

Each aliquot (2 mL tube) then had 75 μL of precipitation enhancer added to it and was inverted 8 times to mix. Then 2 DNA capture beads were added to each aliquot (2 mL tube). Each aliquot (2 mL tube) then had 275 μL isopropanol added to it, and using a vertical rotating mixer, each was mixed at room temperature for 8 minutes at 10 rpm. After incubation, a 1 mL pipette was used to carefully remove all the liquid as far away from the DNA capture beads as possible. Then 500 mL of wash buffer was added to each aliquot (2 mL tube) with DNA capture beads, inverted 4 times. Then a 1 mL pipette was used again to remove all the liquid away from the DNA beads as carefully as possible. The process of adding 500 mL of wash buffer to each aliquot, inverting it 4 times, and carefully removing the liquid away from the beads using a 1 mL pipette was repeated 2 more times.

The DNA collection beads from each aliquot (2 mL tube) were added to the Bead Retainer and pulse-spun to remove any remaining liquid from the DNA Collection Beads. The beads in the Bead Retainer were then transferred to a fresh 2 mL tube and 200 μL of Elution Buffer I was added before incubating at 56° C. for 5 minutes. After incubation, the 2 mL tube of beads and elute were centrifuged for 1 minute at 12,000 rcf. Then using a wide bore 200 pL tipped pipette, each was pipetted about 8 times to disperse any DNA aggregates before storing each at 4° C.

### 2. Preparation of the DNA Mix and PCR Master Mix

A solution of extracted DNA to be used was calculated based on the determination of the number of cells to be evaluated and the estimated concentration of cells in the extracted DNA and kept on ice.

For every 2 μL of sample, a PCR master mix was prepared consisting of:
25.5 μL of Nuclease-free PCR water
8 μL of 5x SuperFi II Buffer
1.6 μL of 10 mM dNTP mix
0.5 μL of 10 μM forward primer
0.5 μL of 10 μM reverse primer
1.6 μL of SuperFi II Polymerase
1.2 μL of 33% PEG 6k
0.4 μL of 100% TWEEN20
0.6 μL of 0.64M 1,2-Propanediol.
   The PCR master mix was then briefly vortexed and kept on ice.

### 3. Droplet Encapsulation and Oil Swap

3 mL syringes with 2% 008-fluorosurfactant (Ran Biotechnology) in Novec-7500 (3M) were prepared. The solution of extracted DNA was combined with the PCR master mix and mixed by gently pipetting with a wide bore 1 mL pipette 8 times. The mixture of extracted DNA solution and PCR master mix was loaded into a 3 mL syringe that was 20% filled with an oil backer of pure Novec-7500. The 200 μL PCR tube strips for collection were prepared by putting them on ice. Then using a microfluidic drop maker, 60 μm diameter droplets were generated. Approximately, 100 μL of the 60 μm diameter droplets were collected into each PCR tube which were then kept on ice for 2 minutes to allow for droplet settling. After settling, the bottom layer of oil was removed and replaced with 5% 008-fluorosurfactant in FC-40 (3M).

### 4. Droplet Thermocycling, Breaking Droplets, and Pooling

The PCR tubes were thermocycled according to the following:

| Step | Temperature | Duration | |
|---|---|---|---|
| 1 | 98° C. | 90 seconds | |
| 2 | 98° C. | 10 seconds | |
| 3 | 60° C. | 10 seconds | |
| 4 | 72° C. | 5 minutes 15 seconds | |
| 5 | | | To Step 2, repeat 34 times |
| 6 | 72° C. | 5 minutes 15 seconds | |
| 7 | 4° C. | ∞ | |

After the thermocycling, PCR strips were kept on ice. Then 10 μL of 1H, 1H,2H,2H-Perfluoro-1-octanol (Sigma) was added to each tube of the PCR strips and left undisturbed for 5 minutes. After 5 minutes, using a wide bore 200 μL pipette, the top aqueous layer from each PCR strip tube was removed carefully to get as much of the aqueous phase as possible. When it was appropriate, the volumes of the aqueous phase were combined into 2 mL tube (DNA low-bind). For each sample the volume was measured using a wide bore 200 μL pipette and the concentration was measured using Qubit fluorometric quantification.

### Example 2: Synthetic Samples Demonstrate a Low-Drop Out Amplification of a Range of Viral Genome Lengths From a Single Tube as Quantified With a Gel

Examples of testing ddLA-PCR's resistance to PCR's propensity to preferentially amplify shorter sequences (kinetic bias) were constructed with a series of plasmid templates carrying either full length JLAT-HIV genome (approximately 9.5 kbp) or truncation lengths within the range of previously reported defective proviruses (6 kbp, 3 kbp, 1 kbp). The control plasmids were used to allow systematic exploration of the ability to recover full length HIV genome even in contexts where truncated proviruses would dominate.

### 1. Improving Droplet Stability by Adding Stabilizing Additives.

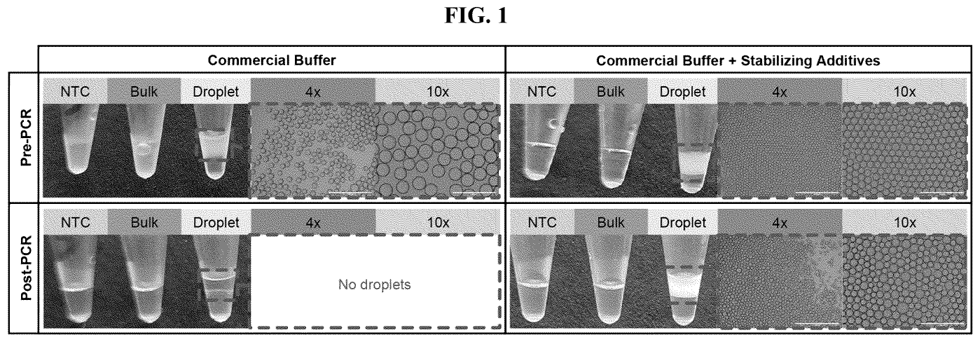

PCR mixes using SuperFi II with and without stabilizing additives were analyzed for droplet stability. Many reactions using only SuperFi II with ddLA-PCR were prone to droplet coalescence or complete catastrophic de-emulsification during the thermocycling process as seen in FIG. 1 under the column labeled “Commercial Buffer.” The same reaction was run using SuperFi II with the additives of PEG 6k MW, TWEEN 20, in an FC40 based oil at concentrations matching values that have been used in conjunction with other PCR mixes. Addition of the additives improved droplet stability after full length HIV amplification reaction as seen in FIG. 1 under the column labeled “Commercial Buffer+Stabilizing Additives.”

2. Optimization of PCR reaction mix.

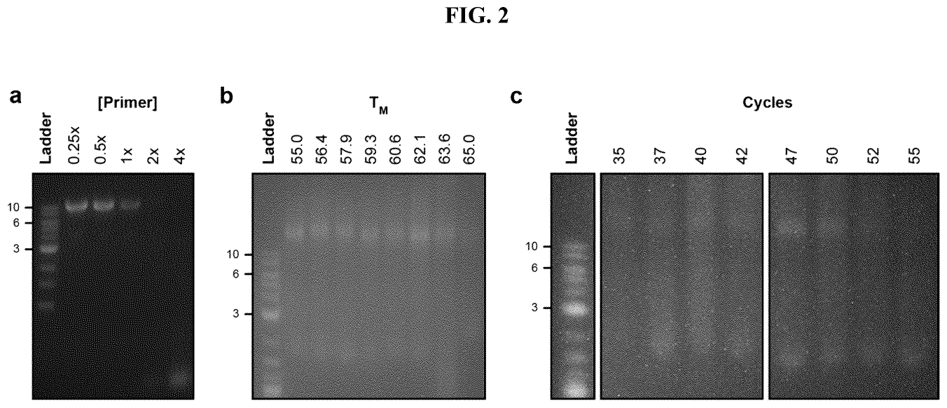

Examples show that for a specific amplicon target, PCR and ddPCR both can greatly benefit from modifying conditions away from the “traditional/generic” PCR mix recommended by a manufacturer. Examples of tests done with alterations in primer, dNTP, and thermocycling parameters identified that decreasing the denaturation temperature offset the known inherent thermal degradation during amplification. Optimized results of modifying the mix with primer alterations is demonstrated in FIG. 2A. Optimized results of primary amplification primers demonstrate robust PCR across a range of anneal temperatures (TM) as demonstrated in FIG. 2B. In addition, examples of the modifications showed that the ddLA-PCR reaction could support up to 50 cycles of amplification as demonstrated in FIG. 2C.

### 3. Optimization of Modified Droplet Based Technique With Multiparameter Analysis of PCR.

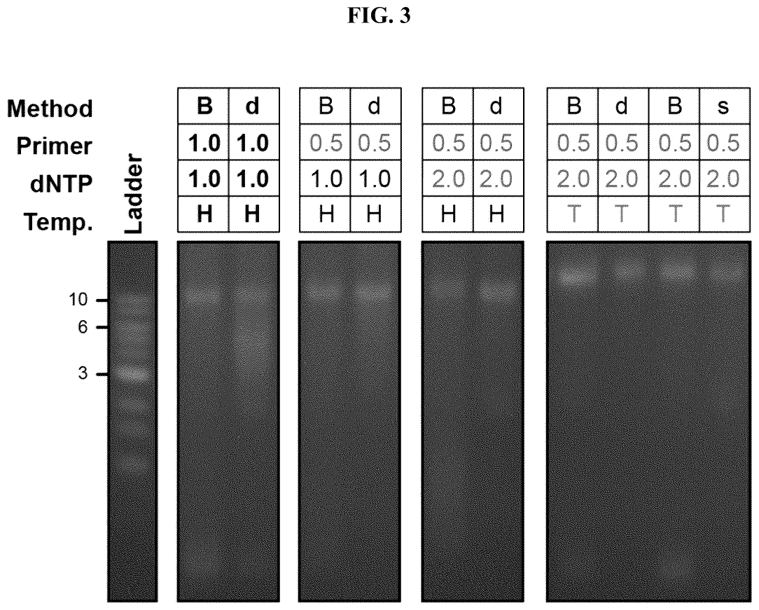

Examples of alterations in primer, dNTP, and thermocycling identified the compatibility of the modified droplet technique with microfluidics and shaken emulsification by showing significant improvement of the sample throughput. See FIG. 3.

### 4. Amplification of Truncated HIV Series Demonstrated by Low-Drop Out Due to High Molecular Weight and Low Concentrations.

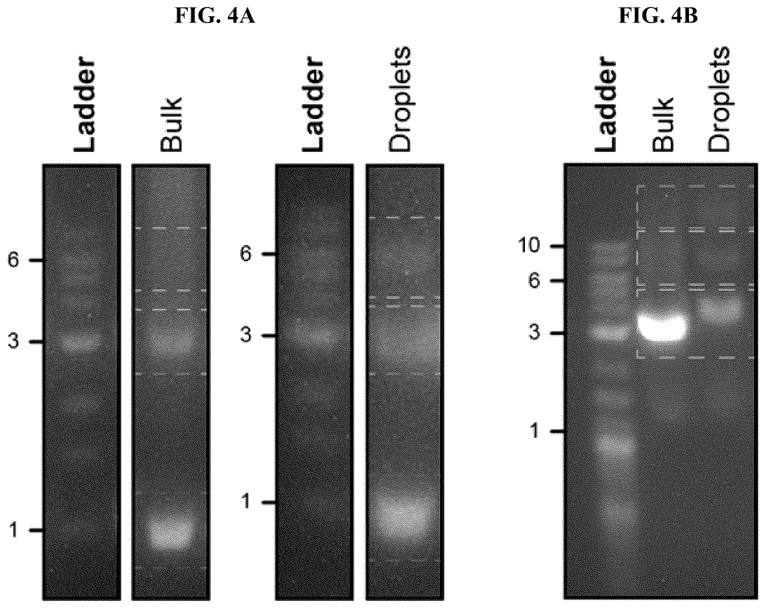

Examples show how the elements of the optimizations were applied to a series of mixture experiments with the truncation series. In the first assay, a ratio of 1:1:1 of 6 kbp, 3 kbp, and 1 kbp provirus plasmids were used. One example showed results performed in bulk and another showed results performed in droplet. The bulk examples showed a heavy preference for the small amplicons, and the larger amplicons either dropped out or became indistinguishable from background noise. The droplet examples showed clear bands identified for the larger amplicons. See FIG. 4A.

A similar test was performed using a ration of 1:1:1 of 10 kbp, 6 kbp, and 3 kbp provirus plasmids. The bulk examples show a heavy preference for the small amplicons, and the larger amplicons either dropped out or became indistinguishable from background noise. The droplet examples showed clear bands identified for the larger amplicons. See FIG. 4B.

### Example 3: Demonstration of the Ability to Sequence Single 10 kb Amplicons Generated From the Tube-Based Reaction

1. Testing the Limit of Detection and the Ability to Run ddLA-PCR Using Excess Background DNA to Particularly Address the Fact That HIV Positive Cells With Replication-Competent Proviruses Typically are Around as Low as 1 in 100,000 CD4+T Cells in ART Patients.

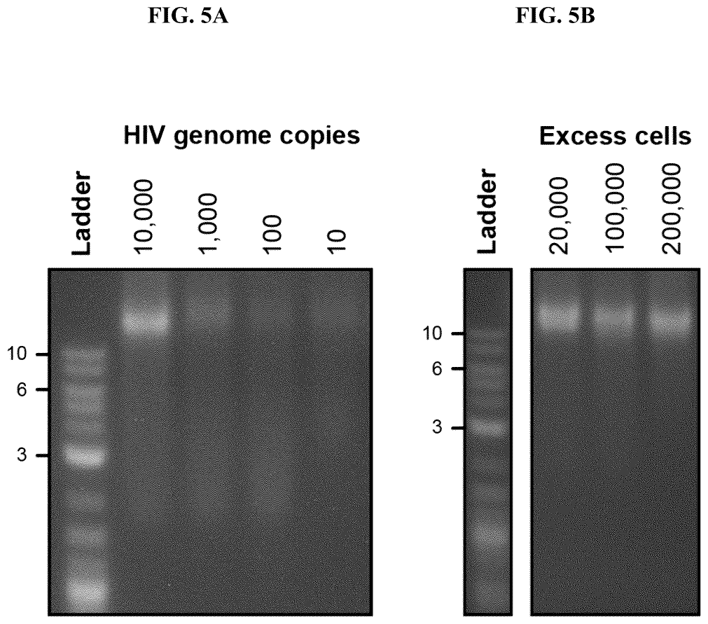

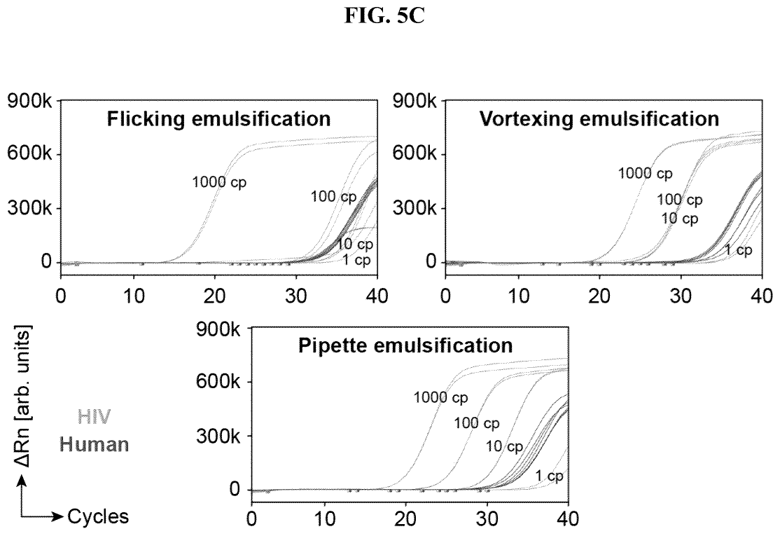

A series of loading experiments were conducted in which the number of HIV copies within the reaction was varied, see FIG. 5A, and the amount of background DNA was varied as seen in FIG. 5B. The results of the experiments confirmed that the ddLA-PCR altered protocol can achieve full length amplification of HIV using real-world anticipated starting concentrations of HIV template based on the super loading of the droplets in a manner similar to what is done in well plate contexts. The examples were performed under the expectations that: a) target number of HIV genomes amplified in the single-tube reaction should be 200-1000 amplified genomes and b) samples from which HIV genomes are amplified should consist of a significant excess of uninfected cell DNA, such as 1 ng total DNA for every HIV genome present in the sample in order to mimic a real sample in which the infection frequency is just below 1 in 100 cells.

Qualitative results from gel electrophoresis were tested and further confirmed with qPCR as part of the determination of the best form of emulsion preparation for the preservation of DNA length for the droplet reaction. Tests were based on observing the signal dynamic range regarding the lower copy-numbers. Tests identified that pipetting produced the most homogenous distribution and/or best preservation of DNA integrity within droplets. See FIG. 5C.

### 2. Optimization of “Second Stage” Reaction.

A “second stage” ddLA-PCR reaction was implemented by using material from the first primary ddLA-PCR reaction because the 10 copies case represents an insufficient level of material for NGS library preparation. The material from the primary ddLA-PCR reaction was used as a template and reamplified in droplets. Examples using this method demonstrated the potential in obtaining sufficient material in the 100s of nanograms that can be utilized for sequencing.

### Example 4: Application of Modification With Samples of Full Length HIV From Patients on ART

### 1. Demonstrating SuperFi II as a Sufficient Storage System.

High-molecular-weight (HMW) DNA is known to be fragile and special handling is required. Intact DNA is a pre-requisite for both LA-PCR and long-read single-molecule sequencing methods like PacBio. Buffer formulations and considerations for handling HMW DNA are already known in the art, including the need for EDTA and high pH. A test was conducted determining the stability of storage at 4° C. as part of ensuring flexibility in the timeline between DNA acquisition and sequencing.

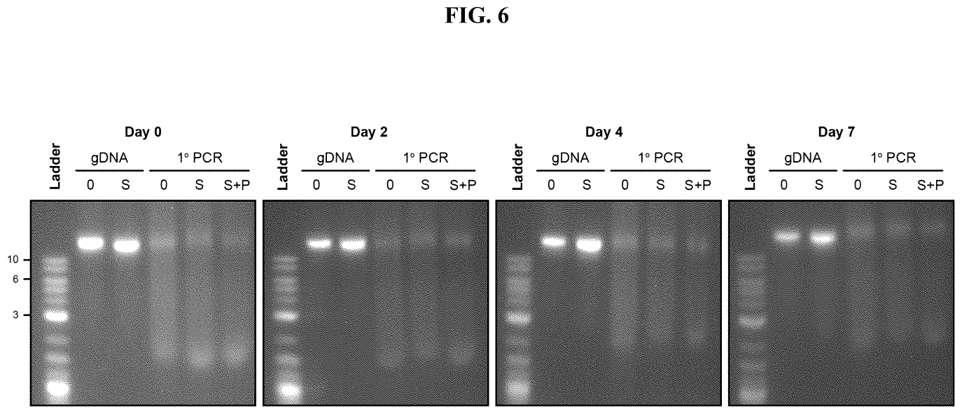

An evaluation of HMW DNA degradation in relevant buffer systems was performed where all samples were stored at 4° C. and aliquots were taken at the indicated times of Day 0, Day 2, Day 4, and Day 7. The evaluation used material that was isolated directly from cells using a New England BioLabs HMW DNA isolation kit and sheered to a similar size as amplicon, which was represented in the results as genomic DNA (gDNA). In addition, the evaluation used amplicons generated from template JLAT gDNA, which was represented in the results as 1° PCR. See FIG. 6. Furthermore, the evaluation involved the comparison of: 1) deionized water for gDNA/SuperFi II buffer for 1° PCR after de-emulsification with a static gun; 2) supplementation of base with a concentrate that leads to a final additional of 10 mM TRIS pH 9.0 and 1 mM EDTA; and 3) supplementation of base with a concentrate that leads to a final additional of 10 mM TRIS pH 9.0 and 1 mM EDTA with a washing with perfluorooctanol for de-emulsification.

The evaluation shows that the stability of HMW DNA at 4° C. with SuperFi II Buffer, thereby identifying SuperFi II Buffer, without augmentation, is a sufficient storage system. See FIG. 6.

2. Isolation and Amplification of Full Length HIV From Patient Samples Using ddLA-PCR.

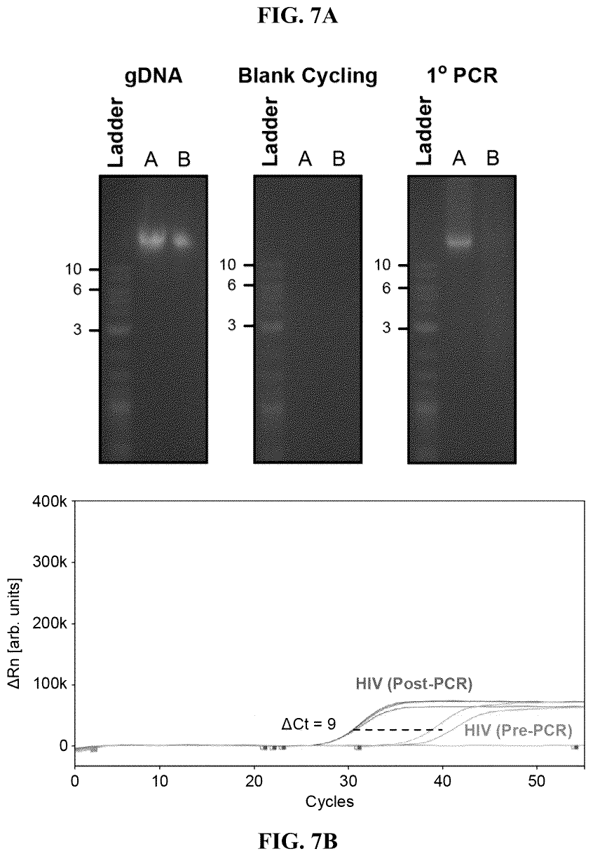

An example of extracting DNA from primary cells and processing them using the same workflow as was used in the JLAT control cell line was demonstrated by isolating and amplifying the high molecular weight DNA from two patient samples. See “A” and “B” in FIG. 7. To permit the highest quality of DNA, a reduced number of cells of 1 million was used for isolation, as opposed to the 5 million maximums. The reduction in cells was shown to reduce the degree of DNA aggregation post isolation, and therefore by removing the need to break up aggregates via pipetting, the amount of HMW DNA shearing was reduced.

The example, as shown in “Blank Cycling” in FIG. 7A, shows that the thermocycling degrades the genomic DNA, but amplification of the HIV patient samples were confirmed with identification as shown in 1° PCR in FIG. 7A for patient sample A. Patient sample B needed to be confirmed, and was confirmed by using qPCR with probes against HIV which was measured at in amplification by approximately 9 Ct (corresponding to approximately 512,000-fold increase). See FIG. 7B.

1. A method for amplifying long target DNA in a sample comprising background DNA, comprising:
a) partitioning DNA from a sample into a plurality of partitions such that, on average, there is one copy of a long target DNA in a partition containing the long target DNA and wherein the partition further comprises background DNA; and
b) amplifying DNA in said plurality of partitions using one or more long target DNA-specific primers under conditions that saturate the partitions containing long target DNA such that all long target DNA in different partitions reach similar endpoint concentrations irrespective of amplification rate.

2. The method of claim 1, further comprising the step of analyzing amplicons generated from said long target DNA.

3. The method of claim 2, wherein said analyzing comprises sequencing said amplicons.

4. The method of claim 1, wherein said long target DNA is obtained from a cell.

5. The method of claim 1, wherein said long target DNA is obtained from a virus.

6. The method of claim 1, wherein DNA from said sample is purified prior to said partitioning.

7. The method of claim 1, wherein said plurality of partitions comprise droplets.

8. The method of claim 1, wherein said partitioning and amplifying are conducted under conditions that avoid fragmenting said long target DNA.

9. A method for analyzing target nucleic acid molecule and sequences flanking said target nucleic acid molecules, comprising: a) fragmenting nucleic acids from a sample such that at least a sub-population of fragments contain full target sequence with a portion of flanking sequence still attached to the target sequence, b) attaching dumbbell primers to the fragments, thereby generating circularized fragments comprising a single stranded loop with partial homology between the primers, c) encapsulating the circularized fragments into partitions, d) amplifying the loops using primers complimentary to portions of the target sequence, thereby generating amplification products that comprise portions of the loops comprising target sequence, flanking regions, and/or loop primers, and e) analyzing amplicons products so as to obtain information about the target sequences and their flanking regions.

10. The method of claim 9, wherein the target nucleic acid is a viral sequence and the sequences flanking the target nucleic acid are mammalian sequences corresponding to a viral sequence integration site in a mammalian genome.

11. The method of claim 9 wherein one or more modified base identifiers is added to a target nucleic acid prior to analysis.

12. A kit comprising reagents sufficient to practice a method of claim 1.

13. A reaction mixture comprising a target nucleic acid and reagents used in the method of claim 9.
